(19) United States
(12) Patent Application Publication
(10) Pub. No.: US 2014/0378515 A1
(43) Pub. Date: Dec. 25, 2014

(54) IMMUNOSUPPRESSIVE COMPOUNDS

(71) Applicants: Muhammad A. Mesaik, Karachi (PK); Khalid M. Khan, Karachi (PK); Omer M. Abdalla, Karachi (PK); Fazal Rahim, Karachi (PK); A. Shukralla Khalid, Karachi (PK); Zaheer-ul Haq, Karachi (PK); Shahnaz Perveen, Karachi (PK); Samreen Soomro, Karachi (PK); Sobia A. Halim, Karachi (PK)

(72) Inventors: Muhammad A. Mesaik, Karachi (PK); Khalid M. Khan, Karachi (PK); Omer M. Abdalla, Karachi (PK); Fazal Rahim, Karachi (PK); A. Shukralla Khalid, Karachi (PK); Zaheer-ul Haq, Karachi (PK); Shahnaz Perveen, Karachi (PK); Samreen Soomro, Karachi (PK); Sobia A. Halim, Karachi (PK)

(21) Appl. No.: 13/921,705

(22) Filed: Jun. 19, 2013

(51) Int. Cl. C07D 277/66

(52) U.S. Cl. CPC C07D 277/66
USPC 514/367; 548/179

(57) ABSTRACT

The invention feature series of benzothiazole derivatives as potent immunosuppressive and antiinflammatory agents. Eight compounds 2, 4, 5, 8, 9, 10, 12, and 18 showed potent inhibitory activity on PHA-activated T-cell proliferation. Compounds 2, 4, 8, and 18 were found to have a potent inhibitory activity with IC₅₀ values ranging <1.0-2.9 μg/mL against IL-2. Studies on innate immune response revealed that compounds 2, 8, 9, and 10 have significant suppressive effect on ROS production with an IC₅₀ values 1.9, <1, 3.7 and 1.1 μg/mL, respectively. The LPS-induced nitrites in J774 macrophages cell line was inhibited by 4, 8, 9, and 18 at a concentration of 25 μg/mL (56-91%). In addition compounds 5, 8, 12, and 18 showed potent suppression on interleukin 4 (IL-4), particularly 9 (IC₅₀<1 μg/mL). No cytotoxicity was found except for compound 9 and 18 (11.4 and 10.4 μg/mL IC₅₀), respectively.

### BACKGROUND OF THE INVENTION

Benzothiazole derivatives, natural or synthetic have been long used as precursors for pharmacological agents and exhibit a variety of biological activities. It is a neuroprotective agent, muscarinic receptor, lymphocyte-specific protein tyrosine kinase (lck) enzyme inhibitor, antibacterial, and antiallergic agents. In this reference, the benzothiazole skeleton constitutes an important central template for a wide variety of biologically active compounds, having many pharmacological functions. Its derivatives are powerful antitumor agents, like phenylbenzothiazoles have been synthesized as pro-drugs and were analyzed for their antitumor activitie and chosen for clinical evaluation. A few derivatives of benzothiazoles especially quinol and 2-(4-aminophenyl) are found to be the most valuable anti-cancer agents both in in vivo and in vitro systems. Recently, a fluorinated 2-arylbenzothiazole was found to have selective and potent inhibitory activity against colon, lung and breast cancer cells. Chromosomal aberrations and gene mutation are involved in cancer growth and inherited clinical disorders. Some amino benzothiazoles derivatives have been recommended to possess mutagenic potency depending on metabolic activation or by inducing DNA damage. Some benzothiazoles derivatives like 2-p-tolyl-benzothiazoles and 2-m-tolyl-benzothiazoles in the T100 strain of Salmonella have mutagenic activity. The benzothiazoles also have immunosuppressive, antiviral, and calmodulin (CaM) antagonist activity.

### BRIEF SUMMARY OF THE INVENTION

In continuation of our ongoing research on the chemistry and bioactivity of new heterocyclic compounds, we reported the β-glucuronidase activity of benzothiazole analogs. We further investigated this class of compounds for the immunomodulatory activity which is disclosed herein. The present work describes the synthesis and evaluation of immunomodulatory potential of a series of benzothiazole derivatives, 1-26, with special emphasis on their effect on the Th-1 cells and on their ability to bind to the Th1 cytokine (IL-2). We have studied the effect of benzothiazole derivatives 1-26 on the phytohemagglutinin (PHA) induced proliferation of T-cell and on IL-2 production by these cells, in order to examine their therapeutic potential on immune system events since the mediators of immune response, when overproduced can cause serious damage including inflammation and injury. Recently, we have efficiently established molecular docking protocols to demonstrate the binding patterns of IL-2 inhibitors at the IL-2 receptor alpha (IL-2Rα) binding site. Using the GOLD docking program, the active IL-2 inhibitors were docked in the ligand binding site of IL-2 protein and their mode of interaction was determined. Furthermore, we have studied the effect of these compounds on the production of a short-lived radical's nitric oxide, end product of the enzymatic oxidation of L-arginine in macrophages and the production of reactive oxygen species, generated by nicotinadenine dinucleotide phosphate (NADPH) oxidase in human peripheral blood phagocytes. In addition to this, the effect of these compounds on IL-4 production and their cytotoxic potential for normal cell line was also monitored.

### BRIEF DESCRIPTION OF THE DRAWING

FIG. 1 depicts the effect of compounds on T-cell Proliferation. PBMNCs were stimulated with 5 μg PHA in the presence of three concentrations of test compounds or without addition of compounds (+ve). Cell proliferation was assessed as indicated in materials and methods section. The figure represent compounds that show potent activity against T-cell proliferation the bars represent mean radioactivity (CPM)±SD of triplicate measurement. Significance difference was calculated in comparison with the PHA positive control (+ve), P<0.005**, P<0.05*. Prs: Prednisolone, −ve: control cells without PHA or compound.

FIG. 2 depicts dose response activity for IL-2 production by PBMNCs. The bars represents the effect of compounds 2, 4, 5, 8 and 18, on IL-2 production using PHA/PMA stimulated PBMCs after 18 h incubation. Data are mean±S.D of the IL-2 produced in μg/mL. P<0.05*, P<0.005**

FIG. 3 depicts the dose response curve for Benzothiazole compounds 4, 8, 9, and 18 on macrophages NO production, stimulated with LPS in the presence of increasing concentrations of the compounds. Results shown are the mean±SD of triplicate measurement expressed as percentage of stimulated control in the absence of compounds.

FIG. 4 depicts the effect of benzothiazole compounds on the production of IL-4, using Human Peripheral Blood Mononuclear Cells (PMBCs). The graph represent effect of seven Benzothiazole compounds on PHA stimulated IL-4 production after 18 hours incubation. Each bar represents a mean of triplicate readings. +C: cells in the presence of PHA,−C: cells alone.

FIGS. 5A-5E depicts the docked binding modes of compounds.

FIG. 5A depicts the interaction of compound 8 with the active site amino acids.

FIG. 5B depicts binding orientation of compound 2 with the active site amino acids

FIG. 5C depicts the binding pattern of compound 18.

FIG. 5D depicts binding orientation of compound 4 with the active site amino acids.

FIG. 5E depicts the docked pose of compound 5 demonstrating that the C3′ substituted OH of compound 5 mediates interaction with the side chain carboxylate (OE1) of Glu68 with the distance of 2.1 Å.

### DETAILED DESCRIPTION OF THE INVENTION

NMR experiments were performed on an Avance Bruker AM 300 MHz machine. CHN analyses were carried out on a Carlo Erba Strumentazion-Mod-1106, Italy. Ultraviolet (UV) spectra were recorded on a Perkin-Elmer Lambda-5 UV/VIS spectrophotometer in MeOH. Infrared (IR) spectra were recorded on a JASCO IR-A-302 spectrometer as KBr (disc). Electron impact mass spectra (EI MS) were obtained on a Finnigan MAT-311A (Germany) mass spectrometer. Thin layer chromatography (TLC) was performed on pre-coated silica gel aluminum plates (Kieselgel 60, 254, E. Merck, Germany). Chromatograms were visualized by UV at 254 and 365 nm or iodine vapors.

In a typical reaction, benzothiazoles 1-26 were synthesized by mixing together commercially available 2-aminothiophenol (3.12 mmol) and different aromatic aldehydes (3.16 mmol) in DMF (10 mL). Sodium metabisulfite Na₂S₂O₅ (0.61 g) was added to a stirring mixture. The reaction mixture was refluxed for 2 h and the progress of the reaction was monitored by TLC. After completion of the reaction, the mixture was allowed to cool to room temperature and water (30 mL) was added. The product was precipitated as a solid, after filtration the benzothiazole derivatives 1-26 were obtained in high yields. Recrystallization from methanol afforded pure products.

Fresh venous blood from a healthy donor was mixed with equal volume of incomplete RPMI-1640 media (Mediatech Inc., Herndon, Va., USA) containing 2 mM L-glutamine and 1% penicillin/streptomycin. The diluted blood was then layered onto lymphocyte separation medium (MP Biomedicals, Inc., Ohio, USA) and centrifuged at 400 g for 20 min at 25° C. The mononuclear cell layer was collected, washed with RPMI-1640 and centrifuged at 300 g for 10 min at 4° C. The peripheral blood mononuclear cells (PBMNCs) were re-suspended in RPMI-1640 complete media containing 10% fetal bovine serum of PAA laboratories GmbH, Pasching, Austria. In a 96-well round-bottomed plate (IWAKI, Scitech. DIV., Ashai Techno glass, Japan), 50 μL of cell suspension (2.5×10⁶ cell/mL), 50 μL, of phytohemagglutinin (PHA) for a final concentration of 5 μg/mL, 50 μL, supplemented RPMI-1640 along with 50 μL of test compounds in a final concentration of 0.5, 5 and 50 μg/mL (in triplicates) were added to the culture mixture. Plates were then incubated at 37° C. in a humidified atmosphere of 5% CO₂ in air for 72 hours. To each well, 0.5 μCi [methyl ³H]-thymidine (Amersham Place Little Chalfont, Buckinghamshire, UK) was added for an additional 18 hours. Cells were harvested using a cell harvester (Inotech, Dottikon, Switzerland), and incorporation and level of radioactivity was measured by a liquid scintillation counter (Beckman coulter, LS 6500, Fullerton, Calif., USA).

The PBMCs were cultured in a 96-well flat-bottomed plate (1.0×10⁵ cell/well) in the presence or absence of three concentrations of test compounds (1.0, 5.0 and 20 μg/mL), and phytohemagglutinin (PHA) phorbol myristate acetate (PMA) in a final concentration of 5 ng/mL and 20 ng/mL, respectively. After an incubation period of 18 h at 37° C. in a humidified atmosphere of 5% CO₂ in air, the supernatant was collected for IL-2 determination. Interleukin-2 levels were measured by using enzyme-linked immunosorbent assay (ELISA) development kit (R&D systems, Minneapolis, Minn., USA). The assay was performed according to the manufacturer's instructions. Briefly, a 96-well flat-bottomed ELISA plate was coated with 4.0 μg/mL mouse anti-human IL-2, in PBS, pH 7.4. Then, re-combinant human IL-2 standards and culture supernatant samples were added and incubated for 2 h followed by washing steps and then the addition of biotinylated goat anti-human IL-2. After an incubation period of 2 h at room temperature, the plates were again washed and streptavidin conjugated horse raddish peroxidase was added and incubated for additional 20 min at room temperature. After the final washes, an enzyme substrate solution of H₂O₂ and tetra methyl benzidine (1:1 v/v) was added and the color was allowed to develop at room temperature in the dark. The plates were then read at 450 nm in a plate reader (DIAReader GMBH, Wr. Neudorf, Austria). The results were analyzed using Microsoft Excel.

Formation of the reactive oxygen species (ROS) in whole blood, during the oxidative burst was measured by the luminol-enhanced chemiluminescence assay. In brief, three concentrations of each compound (1.0, 10 and 100 μg/mL) were prepared in 25 μL of Hank's Balanced salt solution containing calcium chloride and magnesium sulfate (HBSS⁺⁺) in a half area 96-well white flat-bottomed plate (Sigma Aldrich, Steinheim, Germany) in a final volume of 100 μL. Then 25 μL of whole blood diluted 1:50 in suspension of HBSS⁺⁺ was added.

Positive and negative controls and blank wells were included. Cells and compounds were incubated for 30 minutes at 37° C., then 25 μL luminol [3-aminophthalhydrazide] (Research Organics Cleveland, Ohio, USA), were added into each well followed by 25 μL of serum opsonized zymosan (Saccharomyces cerevisiae origin) purchased from Fluka, Buchs (Switzerland) was added except for negative and blank wells. The ROS chemiluminescence kinetic was monitored with a luminometer from Labsystems (Helsinki, Finland), for 50 minutes in the repeated scan mode. Peak and total integral chemiluminescence readings were expressed in the relative light unit.

The mouse macrophage cell line J774.2 obtained from ECACC, Salisbury, Wiltshire (UK) was cultured in T75 flasks in complete DMEM containing 10% fetal bovine serum and supplemented with 1% streptomycin/penicillin. Flasks were kept at 37° C. in the atmosphere of humidified air containing 5% CO₂. Cell were then seeded in 24 well (10⁶ cells/mL) and nitric oxide synthase (NOS-2) in the macrophages was induced by addition of 20 μg/mL E. coli lipopolysaccharide (DIFCO Laboratories, Michigan, USA). The test compounds were added at a concentration of 25 μg/mL soon after LPS stimulation and incubated at 37° C. in 5% CO₂. Cell culture supernatant was collected after 24 hours. Nitrite accumulation in J774.2 cell culture supernatant was measured using the Griess method, where 50 μL of 1% sulphanilamide in 2.5% phosphoric acid, followed by 50 μL of 0.1% naphtyl-ethylene diamine dihydrochloride in 2.5% phosphoric acid were added to 50 μL culture medium. After 10 minutes of incubation at room temperature the absorbance at 550 nm was read. Micromolar concentrations of nitrite were calculated from standard curve constructed with sodium nitrite a reference compound. The results were expressed as means±SD of duplicate readings.

The PBMNCs were cultured in a 96-well flat-bottomed plate (2.0×10⁶ cell/mL) in the presence or absence of three concentrations of test compounds (1.0, 5.0, and 25 μg/mL), and phytohemagglutinin (PHA) in a final concentration of 7.5 μg/mL. After an incubation period of 18 h at 37° C. in 5% CO₂, supernatant was collected for IL-4 determination. Interleukin-4 levels were measured by using enzyme-linked immunosorbent assay (ELISA) kit (Diaclone, France). The assay was performed according to the manufacturer's instructions. Briefly, in a 96-well ELISA plate which was coated with monoclonal mouse anti-human IL-4, 100 μl recombinant human IL-4 standards and culture supernatants samples were added. The plates were incubated for 2 hours at room temperature and washed three times with wash buffer, followed by the addition of biotinylated goat anti-human IL-4 and further incubated for 1 hour at room temperature. The plates were again washed and streptavidin conjugated horse raddish peroxidase was added and incubated for an additional 20 minutes at room temperature. After three final washes, the enzyme substrate solution was added and incubated for 12 to 15 minutes to allow color development at room temperature in the dark. Then stop solution was added and the plates were read at 450 nm in a plate reader (DIAReader GMBH, Wr. Neudorf, Austria). The results were analyzed using Microsoft Excel.

In vitro cytotoxicity assays were performed as described previously, using the 3T3 NIH mouse embryo fibroblast cell line (American Type Culture Collection ‘ATCC’, Manassas, Va. 20108, USA). The 3T3-NIH cells were suspended in Dulbecco's Modified Eagle's Medium (DMEM) formulated with 10% FBS. Using flat bottomed plates, cells were plated at a concentration of 6×10⁴ cells/mL and incubated for 24 h at 37° C. and 5% CO₂ environment. After the removal of media, the cells were challenged with three different concentrations (0.5, 5.0, and 25 μg/mL) of compounds in triplicates and were then further incubated for 48 h at 37° C. in CO₂ incubator. Following exposure to each compound, cell viability was assessed by using 0.5 mg/mL of MTT {3-(4,5-dimethylthiazol-2-yl)-2,5-diphenyltetrazolium bromide for 4 h followed by the removal of supernatant and addition of DMSO to solubilize the formazan complex. The plates were read at 540 nm after one minute shaking and readings were processed using MS Excel software. The results were expressed as means±SD of triplicate readings.

Recently we have reported an efficient molecular docking protocol to predict the binding modes of IL-2 inhibitors using GOLD docking suite and in the current study the same protocol was utilized. To perform docking calculations crystal structure of the Interleukin-2 in complex with phenylalanine methyl ester derivative, (PDB entry code 1M48) was retrieved from the RCSB Brookhaven Protein Data Bank. All water molecules were removed from the original PDB crystal structure. Hydrogen atoms were added to the protein using Biopolymer module in SYBYL 7.3 software package. Ligand structures were constructed by using ChemDraw and converted them into 3D using babel 2.2. Geometry optimizations for the 3D structures were performed using Tripos force field with a distance-dependent dielectric and the Powell conjugate gradient algorithm. Gasteiger-Huckel charges were used for the ligands. GOLD Score was chosen as a fitness function and the standard default settings were used in all calculations. Default cut off values of 2.5 Å for H bonds and 4.0 Å for van der Waal interactions were employed. A 10.0 Å radius active site was drawn on the original position of the co-crystallized ligand and automated cavity detection option was used. After docking thirty poses were saved for each ligand.

Synthesis of benzothiazoles 1-26 were carried out by reacting 2-aminothiophenol with different aromatic aldehydes in N,N-dimethylformamide. In this reaction, sodium metabisulfite (Na₂S₂O₅) was added to a 2-aminothiophenol (3.12 mmol) and different substituted aromatic aldehydes (3.16 mmol) stirring mixture in DMF under reflux for 2 h. The progress of reaction was monitored by TLC. After the completion, the reaction mixture was allowed to cool to room temperature; addition of water (30 mL) precipitated a solid, followed by filtration afforded benzothiazole derivatives 1-26 in good yields (Scheme-1). Recrystallization from methanol afforded pure products (Table-1). The structures of compounds 1-26 were determined by using different spectroscopic techniques including ¹H NMR and EI mass spectroscopy.

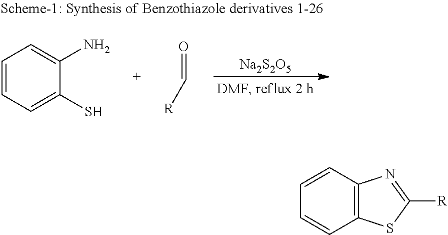

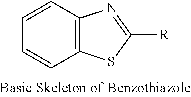

TABLE-1
The structures of compounds 1-26

| Comp. | |
|---|---|
| No. | R |
| 1 | 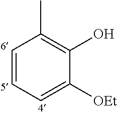 |
| 2 | 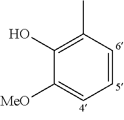 |
| 3 | 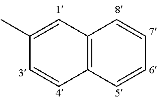 |
| 4 | 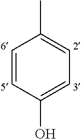 |
| 5 | 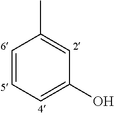 |
| 6 | 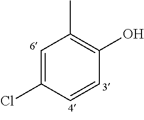 |
| 7 | 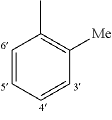 |
| 8 | 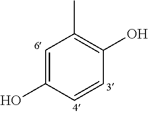 |
| 9 | 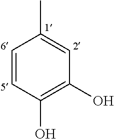 |
| 10 | 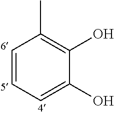 |
| 11 | 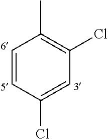 |
| 12 | 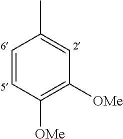 |
| 13 | 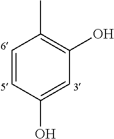 |
| 14 | 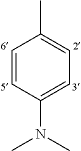 |
| 15 | 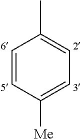 |
| 16 | 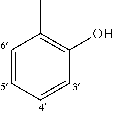 |
| 17 | 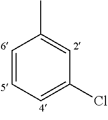 |
| 18 | 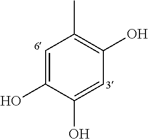 |
| 19 | 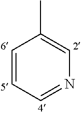 |
| 20 | 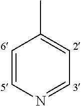 |
| 21 | 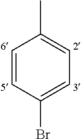 |
| 22 | 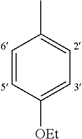 |
| 23 | 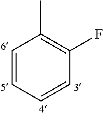 |
| 24 | 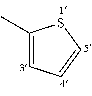 |
| 25 | 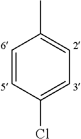 |
| 26 | 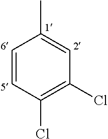 |

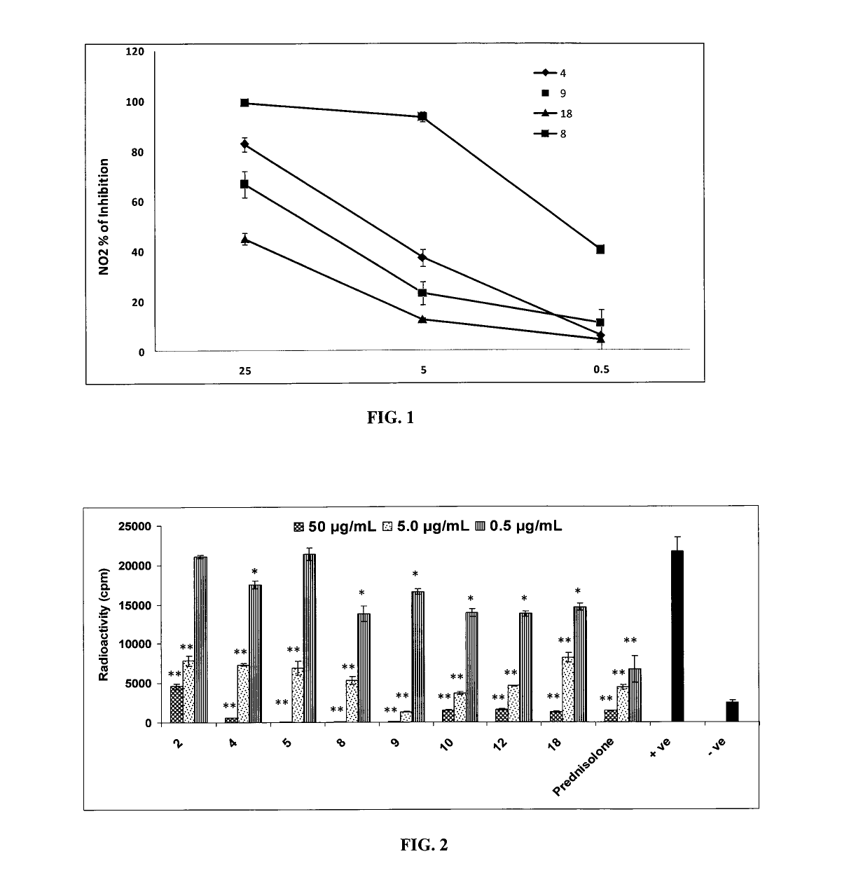

To test for the effect of benzothiazole compounds on T-cells, by performing T-cell proliferation assay. Eight compounds 2, 4, 5, 8, 9, 10, 12 and 18 showed significant suppressive activities (P<0.005) compared to the positive control at a dose of 5.0 and 0.5 μg/mL with IC₅₀ of 2.97±0.3, 2.3±0.1, 2.7±0.2, 1.1±0.1, 1.2±0.0, 0.99±0.1, 1.0±0.1 and 1.95±0.4, respectively. All these eight compounds demonstrated activities comparable to standard drug prednisolone. The inhibition was found to be dose-dependent as shown in FIG. 1. These results indicate that these, compounds having a highly significant inhibitory activity on T-cell immune response and might be useful in various immune disorders.

The ability of some of the selected compounds 2, 4, 5, 8 and 18 that were active in T-cell proliferation assay on production of IL-2 which is the main T-cell growth factor was also evaluated. All five compounds were found to suppress the IL-2 production significantly (p<0.005, <0.05) when compared to the positive control (activated cells without addition of compounds). Four compounds 2, 4, 8 and 18 were found to have a potent inhibitory activity with IC₅₀ ranging between <1.0-2.9 μg/mL. However, only compound 5 was having a slighter lower level of inhibition compared to the afore-mentioned compounds with IC₅₀ of 6.0 μg/mL (FIG. 2).

Compounds 1-26 were screened for immunomodulatory (ROS) activity. Out of twenty-six (26) screened compounds only eight compounds showed significant inhibitory activity against ROS, compound 2, 8-10 had IC₅₀ values of 1.9±0.6, <1, 3.7±0.2 and 1.1±0.1 μg/mL, respectively. These are better than the standard control-(Ibuprofen) (IC₅₀=11.8±1.2 μg/mL) while the compounds 13, 14, 20, and 24 having IC₅₀ values of 11.1±0.9, 11.8±1.2, 15.5±1.0 and 14.4±2.5 μg/mL respectively, also showed moderate inhibitory activity (Table-2). The remaining compounds were found to be inactive as they showed less than 50% inhibition. These results demonstrate that the compounds could potentially exert an inhibitory effect on innate immune response.

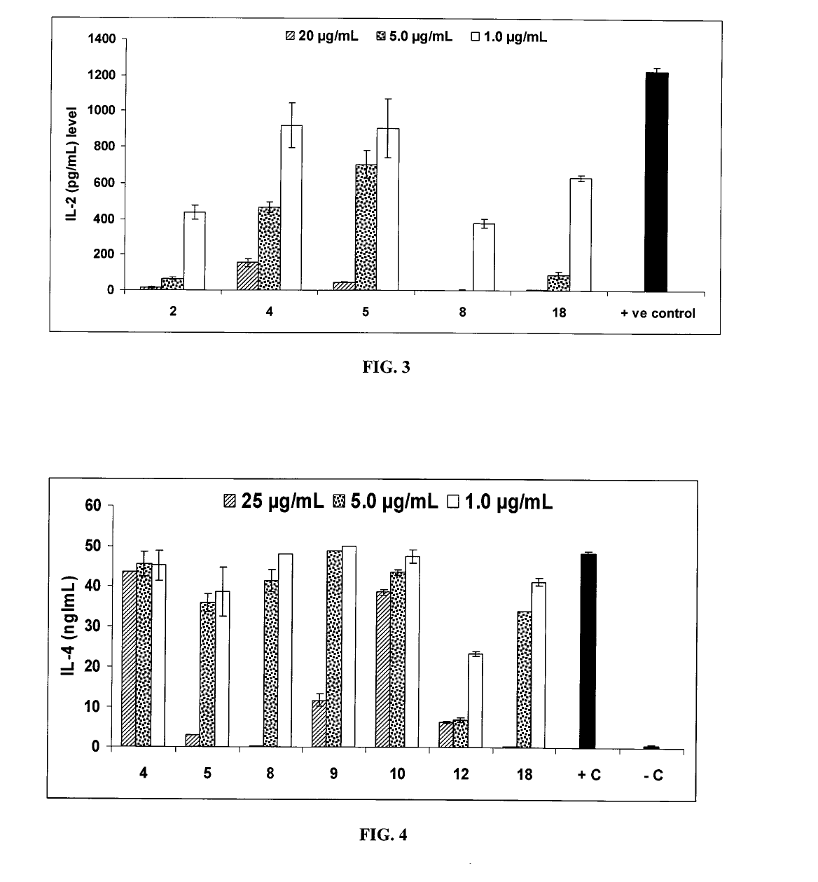

Effect of these compounds on nitrite accumulation by stimulated macrophages was also monitored. LPS treated J774.2 mouse macrophage cell line has been widely used to study the mechanisms of nitric oxide synthase (NOS-2) induction. Here, we studied the effect of benzothiazole compounds on nitric oxide production, and we found that out of twenty-six (26) compounds, four compounds, 4, 8, 9 and 18 showed very potent inhibition against NO with percentage of inhibition of 56.1±0.6, 91.1±0.7, 58.5±3.3 and 78.1±1.6 at 25 μg/mL concentration (Table-2 and FIG. 3). Furthermore, the IC₅₀ for compounds 4, 8, 9 and 18 was found to be 7.9±0.9, <5, 12.6±2.1 and 32.7±4.3 μg/mL, respectively. This NO if over produced then react with superoxide and give rise to a very toxic radical peroxynitrite, which may play a role in many diseases with an autoimmune etiology, so the suppression of NO could be a better target toward inflammatory diseases.

In order to study the target specificity of the compounds that inhibit the T-cell proliferation, the Th-2 cytokine (IL-4) production was measured. Only compound 12 showed potent inhibitory activity with an IC₅₀ of <1.0 μg/mL, while compound 5, 8 and 18 showed moderate inhibitory activities with IC₅₀ of 8.9±0.6, 9.8±0.6 and 8.0±0.0 μg/mL, respectively, (FIG. 4). As observed in the IL-2 assay compounds 2, 4, 5, 8 and 18 which suppressed the production of the IL-2, did not show the similar inhibitory activity against the IL-4 production which reflect that these compounds are specifically inhibit the Th-1 cytokine (IL-2) rather than randomly inhibiting both Th-1 and Th-2 cytokines.

MTT assay was performed to exclude the false positive cytotoxic effect of those compounds, using normal mouse fibroblast cell line 3T3 NIH. We observed that all of the compounds tested, 2, 4, 5, 8, 9, 10, 12, 13, 14, 24 did not interfere with the growth of 3T3 cells up to 20 μg/mL. However, only compound 9 and 18 showed cytotoxic activities with an IC₅₀ of 11.1 and 10.4 μg/mL. Therefore we can assume that benzothiazole derivatives can act as anti-inflammatory lead agents with minor toxicity at higher concentration.

Docking is the most widely used computational tool to identify protein-ligand interactions. We conducted molecular docking to gain insights into the molecular mechanism of protein-ligand binding. The docking performance of GOLD has been validated by re-docking experiments earlier and GOLD docking program was found to be suitable for the docking of IL-2 inhibitors hence GOLD was used to determine the binding orientation of active IL-2 inhibitors into the ligand binding site. The cytokine IL-2 binds with heterotrimeric receptor complex consisting of α, β and γ chains (IL-2Rα, IL-2β and IL-2Rγ). Reported studies suggest that antibodies that block IL-2-IL-2Rα binding have effective clinical implication. Thus the new IL-2 inhibitors was targeted to the IL-2Rα binding site which is dissected into two distinct sub-sites; the first is relatively fixed and includes the center of helix B and the A′-B loop and the other is highly mobile, which comprises the C-terminal end of helix B, the loop connecting helices A and A′, and the loop connecting helix B to helix C.

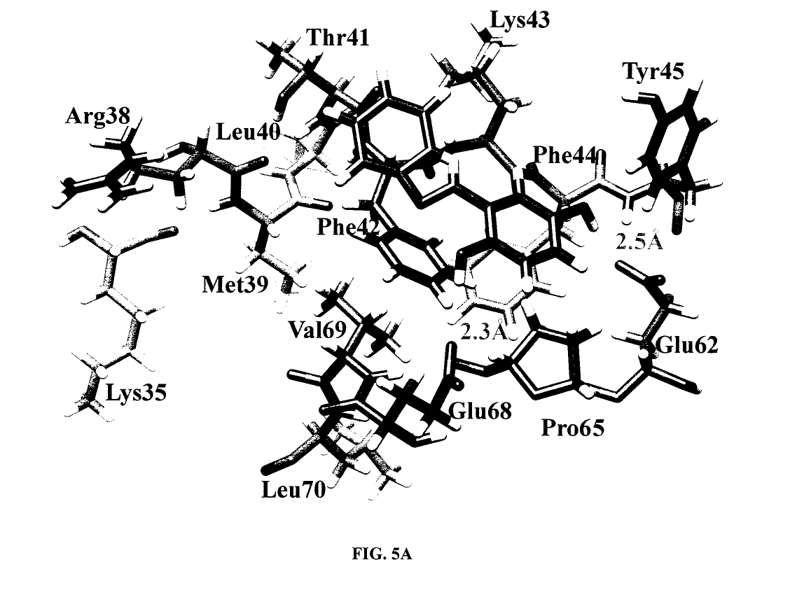

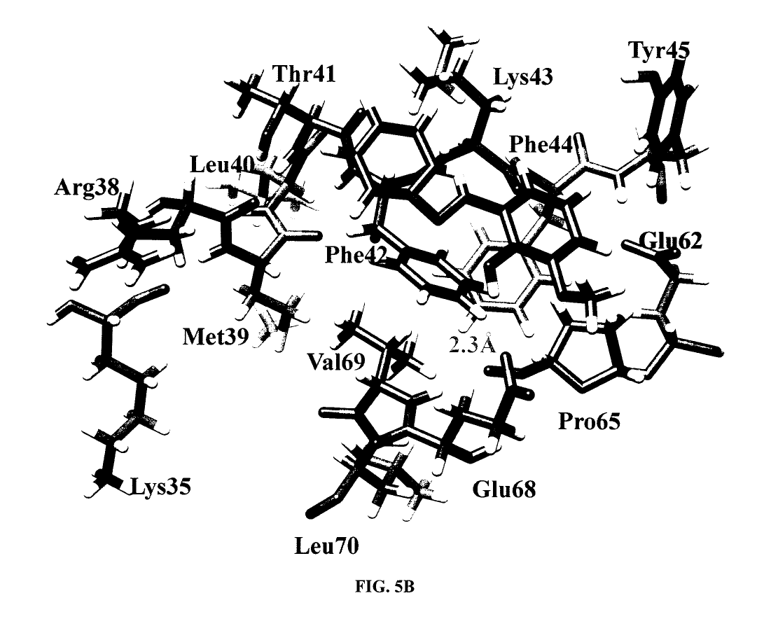

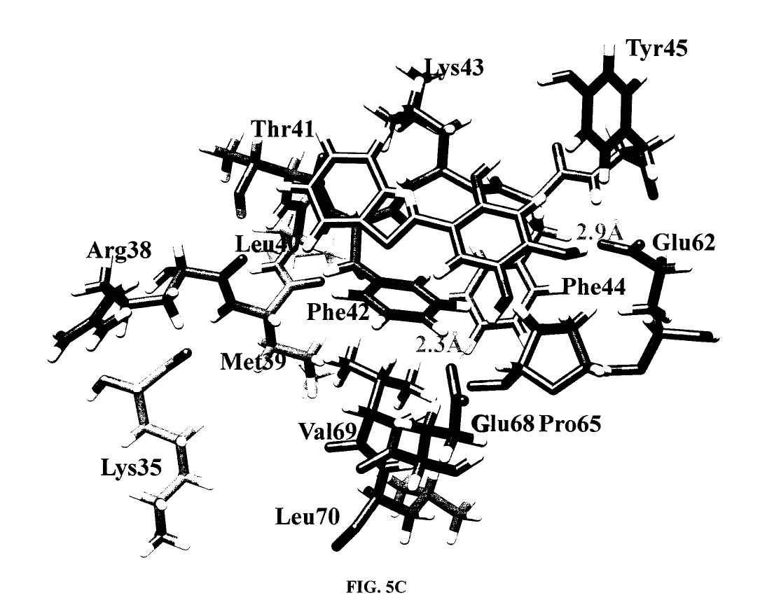

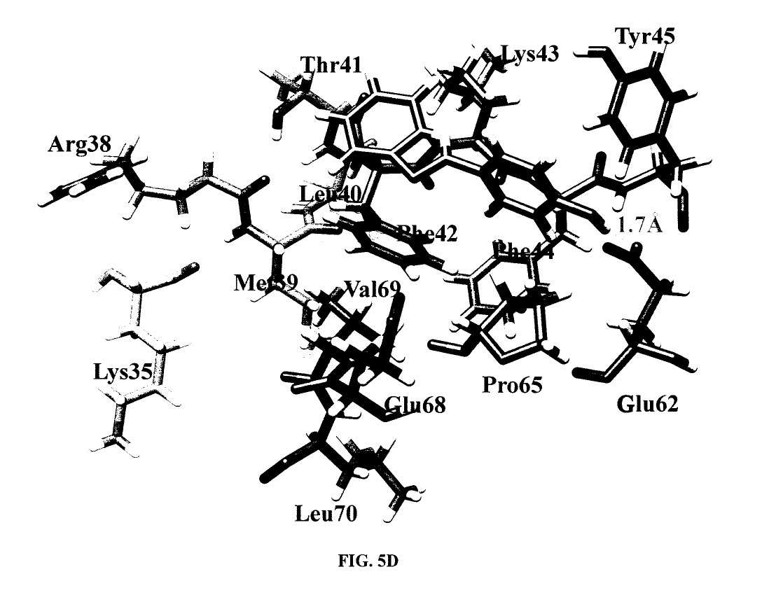

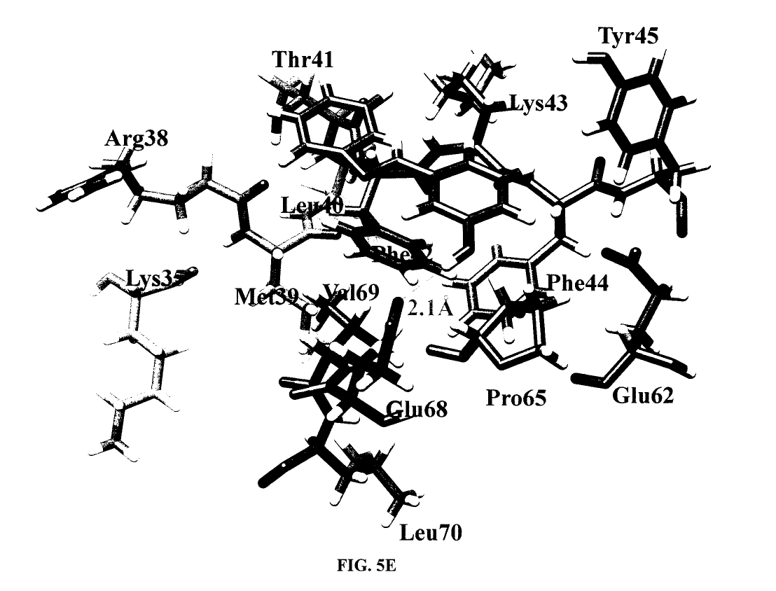

The docked binding modes of compounds 2, 8, 18, 4 and 5 reveals that all these compounds fits into the rigid binding site consisting of Lys43, Tyr45, Glu62 and Glu68. The docking poses of the active compounds reflected that Glu62 and Glu68 are important for hydrogen bonding with the benzothiazole substituted phenol ring. Moreover, two important amino acid residues from the adaptable region (sub-site 2) Phe42 and Phe44, plays important role in stabilization of these compounds by providing π-π interactions. The docked binding modes of compounds are shown in FIGS. 5A-5E. The calculated GOLD scores of compounds 2, 4, 5, 8, and 18 are tabulated in Table 3 which reflects an excellent correlation between docking and experimental data. The predicted correlation coefficient (r²) of docked scores and experimental IC₅₀ was found to be 0.9, which shows that GOLD efficiently predicts the binding pattern of IL-2 inhibitors.

TABLE 3
The GOLD Predicted Docking Scores of Compounds 2, 8, 18, 4 and 5.

| S. No. | Compounds | IC₅₀ (μg/ml) | GOLD Score |
|---|---|---|---|
| 1 | 2 | <1 | 45.82 |
| 2 | 8 | <1 | 45.81 |
| 3 | 18 | 1.1 | 44.42 |
| 4 | 4 | 2.9 | 43.40 |
| 5 | 5 | 6 | 42.15 |

The docked orientation of the most active IL-2 inhibitor (8) reveals that the C2′ hydroxyl group of compound 8 was engaged in hydrogen bonding with the side chain carboxylate (OE 1) of Glu68. While the C5′ substituted OH is hydrogen bonded to the side chain carboxylate (OE1) of Glu62. The hydrogen bond distance between C2′ OH-Glu68 (OE1) and C5′ OH—Glu62 (OE1) was 2.5 Å and 2.3 Å, respectively. The benzothiazole moiety is further stabilized by the π-π interaction provided by the side chain of Phe42, while the side chain of Phe44 provides π-π interaction to the ortho-meta substituted phenol ring. These multiple hydrogen bonds and hydrophobic interaction stabilizes the compound at the IL-2 receptor a (IL-2Rα) binding site of IL-2 protein. The interaction of compound 8 with the active site amino acid residues are shown in FIG. 5A.

As observed in FIG. 5B, the binding pattern of compound 2 is similar to the binding orientation of compound 8 at the IL-2Rα binding site. Similar to the C2′ substituted hydroxyl group of compound 8, the C2′-OH of compound 2 mediates hydrogen bonding interaction with the side chain carboxylate (OE1) of Glu68 with the distance of 2.3 Å. While the C3′ substituted methoxy group does not interact with the surrounding amino acid residues and remains surface exposed. The benzothiazole and the ortho-meta substituted R chain are further stabilized by the π-π interaction provided by the side chains of Phe42 and Phe44, respectively.

The docked pose of compound 18 reflected that the C4′ and C5′ substituted hydroxyl groups of compound 18 mediates interaction with the side chain carboxylate (OE2) of Glu62 and the side chain carboxylate (OE1) of Glu68 with the distance of 2.9 Å and 2.3 Å, respectively. While the C2′ substituted hydroxyl group does not interact with the surrounding residues and remains surface exposed. Similar to the docked conformation of compound 8 and 2, the benzothiazole and the ortho-para-meta-substituted R chain are stabilized by the π-π interaction provided by the side chains of Phe42 and Phe44, respectively. The binding pattern of compound 18 is depicted in FIG. 5C.

From the binding orientation of compound 4, it was depicted that the C4′ substituted hydroxyl group of 4 creates hydrogen bond with side chain carboxylate (OE2) of Glu62. The observed hydrogen bond distance between the C4′OH of 4 and OE2 of Glu62 was 1.7 Å. The side chains of Phe42 and Phe44 provides π-π interaction to the benzothiazole and the ortho-para-meta substituted R chain of compound 4. As shown in FIG. 5D, the lesser activity of the compound 4 as compared to compound 2, 8 and 18 could be due to the lack of hydrogen bond donor substitution at C2′, C3′ and C5′ position of the R chain.

The docked pose of compound 5 (FIG. 5E) demonstrates that the C3′ substituted OH of compound 5 mediates interaction with the side chain carboxylate (OE1) of Glu68 with the distance of 2.1 Å. While the hydrophobic interaction is provided by the side chains of Phe42 and Phe44 as observed in binding orientation of compound 2, 8, 4 and 18. The lack of OH group at position C4′ and C5′ makes the compound 5 lower active, compared to other active hits. The substitution of the hydrogen bond donor at the ortho-meta-para position of the R chain can be favorable for the inhibitory activity of compounds. The docking results shows that the compounds mainly interact with the side chains of Glu62 and Glu68 hence the substitution of either bulky groups or hydrogen bond acceptors are unfavorable at this position and will decrease the inhibitory activity of these compounds.

This study has shown that eight (8) compounds out of the twenty-six (26) benzothiazole derivatives investigated has various activities as a new anti-inflammatory drug lead, where compounds 2, 4, 5, 8 and 18 were found to inhibit the proliferation of T-cells and their production of IL-2 cytokine. For the clear understanding of the inhibition of IL-2 protein, the IL-2 active inhibitors were docked into the IL-2Rα binding site. The docking reveals that the substituted phenol moiety of the compounds interacts with the side chains of Glu62 and Glu68. The hydrogen bond donor groups are favorable for the inhibitory activity while the substitution of bulky groups or hydrogen bond acceptors can lead to significant decrease in activity. The inhibition of oxidative burst and IL-4 production by compound 2, 4, 8, 9, 10 and 12 could be due to inhibition of enzymes involved in their generation for example NADPH oxidase and (iNOS) inducible nitric oxide synthase that are key enzymes in generation of ROS and NO, respectively, or may be due to their inhibitory effect on intracellular signaling molecules.

TABLE 2
Effect of Benzothaizole Compounds on Phagocyte
Oxidative Burst Activity and Nitrite Production. ROS
produced by human blood phagocytes and NO produced
by mouse macrophages J774.2 cell line were determined
as described in material and methods section. Results
are presented as means ± SD of triplicate
measurements.

| | | % of NO | |
|---|---|---|---|
| | IC₅₀ (μg/mL) for | inhibited by 25 μg/mL | Cytotoxicity |
| Compounds | phagocytes ROS | of compound | IC₅₀ (μg/mL) |
| 1 | >100 | 26.5 ± 0.0 | ND |
| 2 | 1.9 ± 0.6 | 28.9 ± 1.3 | >25 |
| 3 | >100 | 20.5 ± 5.9 | ND |
| 4 | >100 | 56.1 ± 0.6 | >25 |
| 5 | >100 | 44.5 ± 0.3 | >20 |
| 6 | 34.3 ± 4.4 | 35.5 ± 7.4 | ND |
| 7 | >100 | 5.4 ± 2.8 | ND |
| 8 | <1 | 91.1 ± 0.7 | >20 |
| 9 | 3.7 ± 0.2 | 58.5 ± 3.3 | 11.4 ± 1.8 |
| 10 | 1.1 ± 0.1 | 14.3 ± 1.0 | ND |
| 11 | >100 | −7.4 ± 3.0 | ND |
| 12 | >100 | −1.5 ± 0.6 | ND |
| 13 | 11.1 ± 0.9 | 32.7 ± 8.3 | 11.5 ± 0.0 |
| 14 | 11.8 ± 1.2 | 16.8 ± 0.8 | >25 |
| 15 | >100 | 10.8 ± 2.4 | ND |
| 16 | >100 | 20.2 ± 1.5 | ND |
| 17 | 81.0 ± 4.1 | 14.1 ± 3.0 | ND |
| 18 | >100 | 78.1 ± 1.6 | 10.4 ± 1.3 |
| 19 | >100 | 11.7 ± 1.7 | ND |
| 20 | 15.5 ± 1.0 | 41.6 ± 4.7 | >25 |
| 21 | >100 | 11.5 ± 2.7 | ND |
| 22 | >100 | 23.5 ± 3.7 | ND |
| 23 | >100 | 28.4 ± 2.5 | ND |
| 24 | 14.4 ± 2.5 | 30.3 ± 0.3 | >25 |
| 25 | >100 | 20.6 ± 5.3 | ND |
| 26 | >100 | 35.7 ± 1.5 | ND |

ND = not determined.

What is claimed is:

1. An anti-inflammatory benzothiazole derivative selected from the group consisting of:
4.1.2. 2-(1,3-benzothiazol-2-yl)-6-methoxyphenol;
4.1.4. 4-(1,3-benzothiazol-2-yl)phenol;
4.1.8. 2-(1,3-benzothiazol-2-yl)-1,4-benzenediol;
4.1.9. 4-(1,3-benzothiazol-2-yl)-1,2-benzenediol;
4.1.10. 3-(1,3-benzothiazol-2-yl)-1,2-benzenediol;
4.1.12. 2-(3,4-Dimethoxyphenyl)-1,3-benzothiazole; and
4.1.18. 5-(1,3-benzothiazol-2-yl)-1,2,4-benzenetriol.

2. The benzothiazole derivative of claim 1, further comprising a pharmaceutically acceptable excipient, diluent and/or carrier.

3. A method of treating an inflammatory disease or disorder comprising: administering to a subject in need thereof a pharmaceutical composition comprising an effective amount of:
4.1.2. 2-(1,3-benzothiazol-2-yl)-6-methoxyphenol;
4.1.4. 4-(1,3-benzothiazol-2-yl)phenol;
4.1.8. 2-(1,3-benzothiazol-2-yl)-1,4-benzenediol;
4.1.9. 4-(1,3-benzothiazol-2-yl)-1,2-benzenediol;
4.1.10. 3-(1,3-benzothiazol-2-yl)-1,2-benzenediol;
4.1.12. 2-(3,4-Dimethoxyphenyl)-1,3-benzothiazole; or
4.1.18. 5-(1,3-benzothiazol-2-yl)-1,2,4-benzenetriol.

4. The method of treating an inflammatory disease or disorder according to claim 3, wherein the inflammatory disease or disorder is sarcoidosis, ankylosing spondylitis, arthritis, osteoarthritis, rheumatoid arthritis (RA), psoriatic arthritis, inflammatory respiratory disease, asthma, atherosclerosis, multiple sclerosis, inflammatory bowel disease, Crohn's disease, colitis, ulcerative colitis, dermatitis, fibromyalgia, systemic lupus erythematous (SLE), nephritis, or Parkinson's disease.

5. The method of treating an inflammatory disease or disorder according to claim 3, wherein the treatment comprises parenteral, enteral or oral administration.

6. The method of treating an inflammatory disease or disorder according to claim 3, wherein the pharmaceutical composition further comprises a pharmaceutically acceptable excipient, diluent and/or carrier.
